(19) United States
(12) Patent Application Publication
(10) Pub. No.: US 2019/0167110 A1
(43) Pub. Date: Jun. 6, 2019

(54) CONTROL PROCESSING SYSTEM AND IMAGING METHOD FOR SUBCUTANEOUS VEIN DEVELOPING DEVICE

(71) Applicant: SHENZHEN UNIVERSITY, Shenzhen, Guangdong (CN)

(72) Inventors: Guo DAN, Guangdong (CN); Zihao CHEN, Guangdong (CN); Yu YI, Guangdong (CN); Siping CHEN, Guangdong (CN)

(73) Assignee: Shenzhen University, Shenzhen, Guangdong (CN)

(21) Appl. No.: 16/304,560

(22) PCT Filed: Oct. 8, 2016
(86) PCT No.: PCT/CN2016/101513

§ 371 Date: Nov. 26, 2018

(30) Foreign Application Priority Data
May 26, 2016 (CN) 201610356998.8

(51) Int. Cl. A61B 5/00; A61B 5/02; G16H 30/40

(52) U.S. Cl. CPC A61B 5/0082; A61B 5/004; A61B 5/02007; A61B 5/1079; A61B 5/489; A61B 5/743; G16H 30/40

(57) ABSTRACT

A control processing system and an imaging method for a subcutaneous vein developing device are provided. The control processing system includes a processor subsystem and a programmable logic subsystem. The processor subsystem and the programmable logic subsystem are interconnected via a high-bandwidth Advanced eXtensible Interface (AXI) bus. The control processing system is in signal communication with a visible light source driving circuit, a near infrared light source driving circuit, a projection imaging element driving circuit, a near infrared imaging element driving circuit, a display screen driving circuit, and a user control interface. For a subcutaneous vein developing imaging application and imaging characteristics thereof, a control processing system architecture of a subcutaneous vein developing system is designed and is implemented in a manner of combining software and hardware.

### FIELD OF THE INVENTION

The present disclosure relates to the technical field of medical instruments, and in particular, to a control processing system and an imaging method for development of venous vessels during subcutaneous venipuncture.

### BACKGROUND OF THE INVENTION

Subcutaneous venipuncture is one of the most common medical care operations in hospitals and is an important means for clinical diagnosis and treatment. However, puncture for obese patients and infant patients has always been a headache for medical staff. According to statistics, subcutaneous venipuncture is performed 1 billion times each year in the United States, with an average of more than 3 times per person per year. In China, subcutaneous venipuncture is performed over 10.4 billion times, that is, eight times per person, which is greater than an average of 2.4 to 3.2 times in the whole world. Target groups of subcutaneous vein puncture in hospitals are generally described as follows. The first group of subcutaneous are patients with acute or serious diseases, which accounts for about 40%. These patients have poor peripheral circulation, resulting in particular difficulty in subcutaneous venipuncture. The second group of subcutaneous are elderly people receiving healthcare treatment, which accounts for about 50%. Blood vessels of elderly people have poor elasticity and increased brittleness. In addition, long-time infusion can cause damage to blood vessels, which becomes a major difficulty in subcutaneous venipuncture. The third group of subcutaneous are infant patients which accounts for approximately 10%. These patients have thin vessels that are difficult to find, causing great inconvenience to subcutaneous venipuncture. In addition, patients and their parents grow increasingly sensitive to multiple times of puncture, puncture misses, or even puncture failures.

Therefore, a subcutaneous vein developing system emerges. However, at present, a commercially available subcutaneous vein developing system usually uses only a single embedded microprocessor or a single programmable logic device. Due to the limitations of a control processing system architecture adopted, some significant delay problems occur when a more complex processing algorithm or more processing operations need to be implemented to achieve a better imaging effect. It is difficult to satisfy increasing requirements in terms of real-time performance and intelligence.

### SUMMARY OF THE INVENTION

To resolve the defects of significant time delays and inadequate intelligence and the like that exist in existing products and increase the imaging quality of a system, the disclosure provides a control processing architecture for a subcutaneous vein developing device, and develops, by focusing on research and implementation of imaging technologies of subcutaneous vein developing, a subcutaneous vein developing system that has higher contrast, lower time delay, and higher intelligence.

Key technologies of the subcutaneous vein developing system designed in the disclosure include technologies such as control processing system architecture, image contrast enhancement processing, or in-situ isometric projection.

To achieve the objectives of the disclosure, the technical solutions used in the disclosure are as follows.

A control processing system includes a processor subsystem and a programmable logic subsystem. The processor subsystem and the programmable logic subsystem are interconnected via a high-bandwidth bus. The control processing system is in signal communication with a visible light source driving circuit, a near infrared light source driving circuit, a projection imaging element driving circuit, a near infrared imaging element driving circuit, a display screen driving circuit, and a user control interface. The high-bandwidth bus is an Advanced eXtensible Interface (AXI) bus, including AXI-Lite and AXI-Stream.

AXI4-Lite is a subset of AXI interfaces and is used for a processor to perform communication with a control register in a device (module). AXI4-Stream is also a subset of AXI interfaces and is used as a standard interface for connecting a device (module) that requires exchange of a large amount of data. AXI-Stream interfaces support many different stream types. AXI-Stream-based video stream type interfaces are used for interfaces of all video processing modules in the system.

The visible light source driving circuit, the near infrared light source driving circuit, the projection imaging element driving circuit, the near infrared imaging element driving circuit, the display screen driving circuit, and the user control interface are in signal communication with the control processing system, to implement data collection and projection imaging of a near infrared image of a subcutaneous venous vessel. The control processing system is responsible for image data processing and system control.

The control processing system further includes an image data collection module, an image cutting and scaling module, an image exposure statistics module, an image contrast enhancement module, a video source multiplexing module, a projection output module, a display screen output module, an automatic exposure adjuster and controller module, an image exposure assessment module, a system parameter control module, and a memory module. The modules are in signal communication with each other.

An image processing method using a control processing system includes the following steps:

A) collecting data on a near infrared image of a subcutaneous venous vessel, and performing cutting, scaling, and offset adjustment processing on a collected image;

B) adjusting a near infrared light source in a subcutaneous vein developing device system according to an exposure condition of the collected image, to provide a stable and appropriate image exposure status for subsequent image enhancement processing, where the adjusting a near infrared light source in a subcutaneous vein developing device system includes image exposure statistics, image exposure assessment, and automatic exposure adjustment and control of the light source;

C) enhancing the contrast of the image; and

D) performing further processing on a resultant image to be output, to implement two-way synchronous display output of a projection imaging element and a display screen.

In the image processing method, in Step A), sizes of actual coverage regions of a collection lens and a projection lens are measured respectively according to a projected image, and a position of the coverage region of the projection lens relative to the coverage region of the collection lens is measured, to perform cutting, scaling, and offset adjustment processing on the collected image.

In the image processing method, in step B), in the image exposure statistics different weight values are set according to degrees of attention given by a user to different regions and then performing weighted averaging on exposure information of each region, and next, comparison and assessment are performed on obtained image exposure information and automatic exposure adjustment and control of the near infrared light source is implemented.

In the image processing method, in step C), an image contrast enhancement method can be a transform domain-based method or a histogram equalization method and various improvement methods derived therefrom.

In the image processing method, the improvement methods include global histogram equalization, or brightness preserving bi-histogram equalization, or Sigmoid function-based bi-histogram equalization, or contrast limited adaptive histogram equalization (CLAHE).

In the image processing method, in step D), an output video is further processed by using a time-division multiplexing method, to implement two-way synchronous display output of the projection imaging element and the display screen.

### Main Beneficial Effects of the Disclosure

The disclosure relies on a design concept in which a processor subsystem and a programmable logic subsystem work in coordination, so that a contrast enhancement technology for a subcutaneous venous vessel image can effectively perform contrast enhancement on a subcutaneous venous vessel and surrounding tissue thereof and can further implement a real-time imaging process.

The disclosure provides a CLAHE-based improvement method, and the control processing system architecture provided in the disclosure is used for design and implementation, so that ideal effects are achieved in both aspects of contrast enhancement of subcutaneous venous vessel image and real-time performance.

The disclosure provides an in-situ isometric projection technology for subcutaneous vein developing and imaging. The technology implements the overlapping of positions of a projected image of a subcutaneous venous vessel and an actual subcutaneous venous vessel.

The disclosure provides an adaptive exposure control technology for subcutaneous vein developing and imaging. The technology adjusts an exposure condition of a subcutaneous venous vessel image automatically, so that the image is kept in a stable and appropriate exposure status, thereby ensuring a stable imaging effect in the presence of external interference.

Finally, the disclosure uses a manner of combining software and hardware for technical implementation. A subcutaneous vein developing system is designed with higher contrast, lower time delay, and higher intelligence, thereby effectively assisting medical staff in positioning a subcutaneous venous vessel of a puncture target and increasing the success rate of a subcutaneous venipuncture operation.

### BRIEF DESCRIPTION OF THE DRAWINGS

FIG. 1 is a schematic block diagram of structural connections of a control processing system according to an embodiment;

FIG. 2 is a schematic diagram of an image collection path and a projection light path being coaxial according to an embodiment;

FIG. 3 is a schematic diagram of an in-situ isometric projection method according to an embodiment;

FIG. 4 is a structural block diagram of an imaging method according to an embodiment;

FIG. 5 is a schematic diagram of imaging region division and weight distribution according to an embodiment;

FIG. 6 is an abstract schematic diagram of an image exposure assessment module and an automatic exposure adjustment and control module according to an embodiment;

FIG. 7 is a specific schematic flowchart of an adaptive exposure control algorithm for a near infrared light source according to an embodiment;

FIG. 8 is a schematic diagram of image pixel reconstruction and mapping according to an embodiment;

FIG. 9 is a schematic block diagram of an image contrast enhancement module according to an embodiment;

FIG. 10 is a schematic block diagram of a histogram statistics module according to an embodiment;

FIG. 11 is a block diagram of a mapping establishment/output module according to an embodiment;

FIG. 12 is a schematic diagram of a flow-line framework of bilinear interpolation according to an embodiment; and

FIG. 13 is a block diagram of a technical implementation of two-way synchronous display according to an embodiment.

### DETAILED DESCRIPTION OF THE EMBODIMENTS

The disclosure is further described below in detail with reference to the accompanying drawings and specific embodiments.

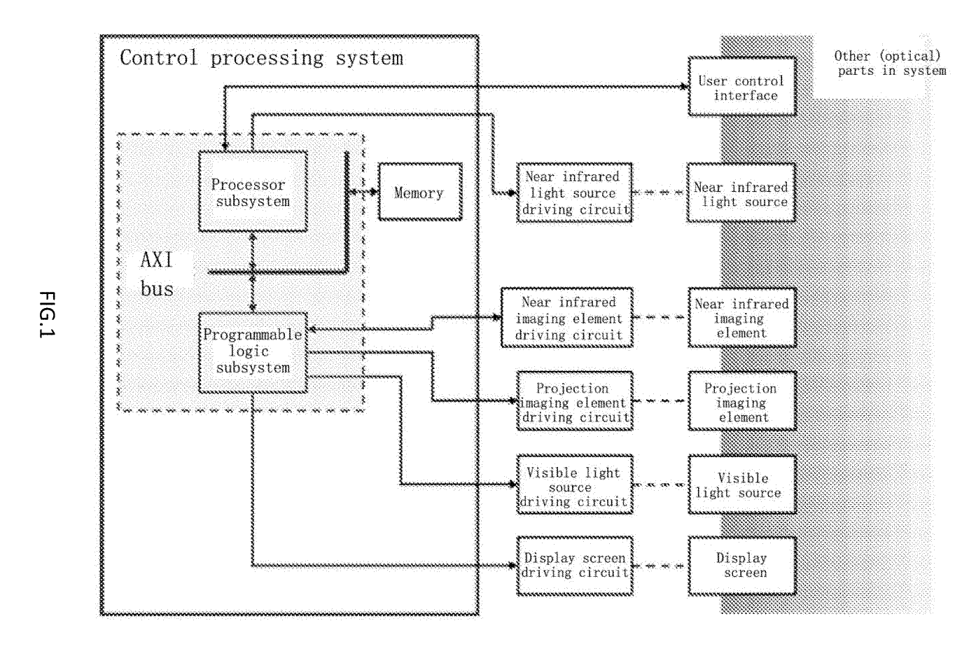

A block diagram of the structure and connection of a control processing system described in the present invention is shown in FIG. 1.

As shown in FIG. 1, the control processing system includes a processor subsystem, a programmable logic subsystem, and a memory. The processor subsystem, the programmable logic subsystem, and the memory are interconnected via an AXI bus. A near infrared light source driving circuit and a user control interface are in signal communication with the processor subsystem. A near infrared imaging element driving circuit, a projection imaging element driving circuit, a visible light source driving circuit, and a display screen driving circuit are in signal communication with the programmable logic subsystem.

The processor subsystem can be implemented as a microprocessor with an ARM architecture or another microprocessor having a similar function. The programmable logic subsystem can be implemented as a field-programmable gate array (FPGA) device or another programmable logic device having a similar function. The memory can be implemented as a double data rate (DDR) memory or another memory device having similar performance.

Furthermore, the processor subsystem, the programmable logic subsystem and the AXI bus can be formed by using a Zynq heterogeneous system-on-chip or another system-on-chip device having a similar function.

The near infrared light source driving circuit, the near infrared imaging element driving circuit, the projection imaging element driving circuit, the visible light source driving circuit, and the display screen driving circuit are respectively in signal communication with a near infrared light source, a near infrared imaging element, a projection imaging element, a visible light source, and a display screen.

Image Cutting and Scaling (In-Situ Isometric Projection)

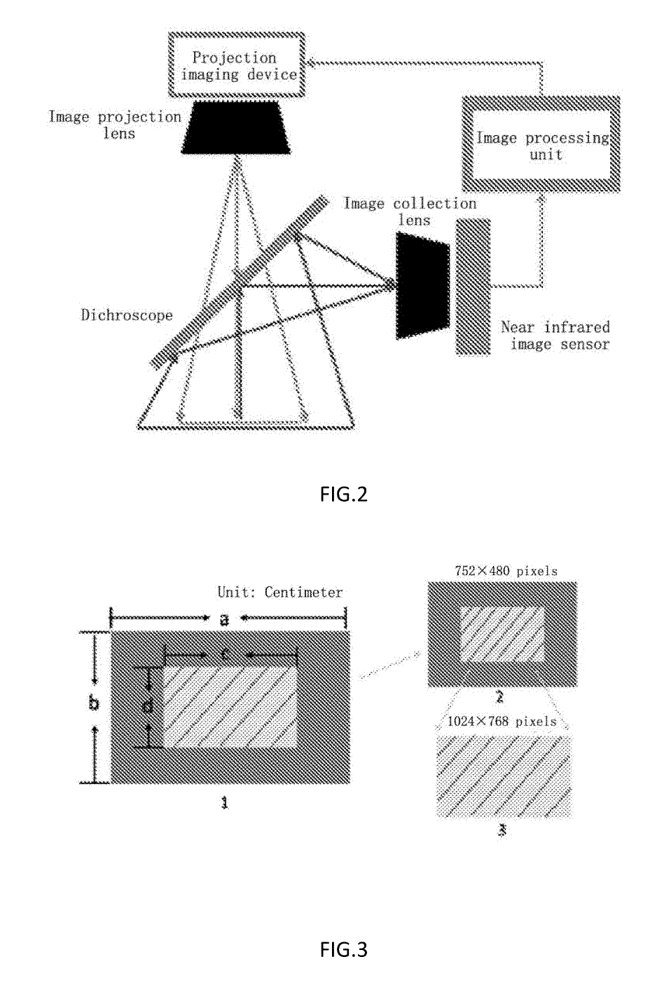

To implement the overlapping of positions of a subcutaneous venous vessel in a resultant projection image and a subcutaneous venous vessel in an actual region of interest, in-situ isometric projection needs to be performed. Coaxial light paths are implemented for optical imaging units in the system by using a dichroscope. That is, the centers of a light path of image collection and a light path of projection are approximately overlapping, as shown in FIG. 2 and FIG. 3.

The light paths of image collection and projection are coaxial, but a collection device and a projection device have inconsistent resolutions and inconsistent field of views (FOV) of lenses. Therefore, to implement in-situ isometric projection, a collected image further needs to be processed. Operations such as image isometric scaling and offset adjustment are mainly included.

In a plane at an effective working height h=30 cm, it is set that a size of an actual coverage region of a collection lens of an image is a×b centimeters, and a size of an actual coverage region of a projection lens is c×d centimeters. To achieve completely isometric overlapping between a projected image and an actual object, a region covered by the projection lens needs to be captured from an image collected by an image sensor. The region is, for example, a slant-line region in the second part shown in FIG. 3. The region is then enlarged to a resolution of a projection imaging device, and is then projected by the projection imaging device, as shown by the third part in FIG. 3. In this case, the projected image can isometrically overlap an object covered by the projected image in actual space.

According to the design solution provided above, specific steps of implementing in-situ isometric projection in the disclosure are as follows:

(1) In a plane at the effective working distance h=30 cm in the system, the collected image is first enlarged to a resolution size of the projection imaging device and is then projected by the projection imaging device. The sizes of the actual coverage regions of the collection lens and the projection lens are measured respectively according to a projected image, and are respectively denoted as a×b=16.3 cm×10.8 cm and c×d=8.8 cm×6.5 cm. Offset amounts x=3.7 cm and y=1.3 cm of the coverage region of the projection lens relative to the coverage region of the collection lens are further measured at the same time.

(2) According to the data measured above, a size and an offset amount of a part that needs to be cut from the collected image are calculated. It is set that a resolution of an image collected by a complementary metal-oxide-semiconductor (CMOS) image sensor is 752×480 pixels, and it is set that the length and width of the part that needs to be cut are respectively m̅ pixels and n̅ pixels, wherein:

$$\begin{matrix}\overline{m} = \frac{c}{d} \times 752 ( pixs ) & ( 1 ) \\ \overline{n} = \frac{d}{b} \times 480 ( pixs ) . & ( 2 )\end{matrix}$$

In addition, it is set that the starting offset coordinate of the cut part is (x̅, y̅), where:

$$\begin{matrix}\overline{x} = \frac{x}{a} \times 752 ( pixs ) & ( 3 ) \\ \overline{y} = \frac{y}{b} \times 480 ( pixs ) . & ( 4 )\end{matrix}$$

According to calculation results, a region whose size is m̅×n̅ and whose starting offset is (x̅, y̅) is captured from the collected image. In this case, the captured image is the region that is covered by the projection lens.

(3) A cutting and enlargement module is designed according to the parameters determined in the previous step. The module uses a video stream interface based on an AXI bus standard. A backward enlargement algorithm is used inside the module to implement the real-time enlargement of the image.

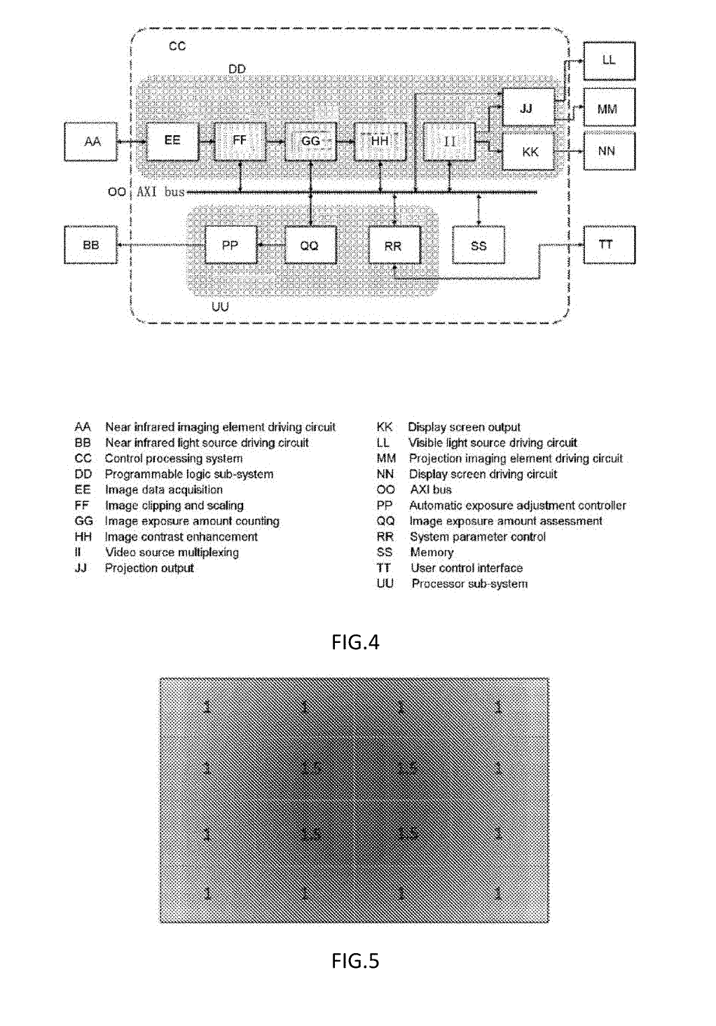

In the disclosure, as shown in FIG. 4, an image cutting and scaling module is used as first-level processing after image data collection to implement an in-situ isometric projection function.

Image Exposure Statistics and Automatic Exposure Adjustment and Control (Adaptive Exposure Control of the Near Infrared Light Source)

A subcutaneous vein developing system designed in the disclosure needs to use the near infrared light source for lighting. In different environments such as an indoor environment or an outdoor environment, a daytime environment or a nighttime environment, and a sunny environment or a cloudy environment, illumination conditions of near infrared light are different. If the illumination of near infrared light is insufficient, the collection of image data is severely affected. If near infrared light is excessively intense, the collected image can be overexposed, and subsequent processing is also affected. It can be seen that to ensure an optimal illumination effect is especially important for the imaging quality of the system.

The adaptive exposure control of the near infrared light source is to ensure that an optimal illumination effect can be acquired for image data collection of the system in different environments, to provide a stable and appropriate exposure status for subsequent image enhancement processing. The adaptive exposure control of the near infrared light source designed in the disclosure is used as second-level processing after image data collection in the system. The near infrared light source in the system is adjusted in real time according to an exposure condition of a currently acquired image. The adaptive exposure control includes three parts: image exposure statistics, image exposure assessment, and automatic exposure adjustment and control for the near infrared light source, as shown in FIG. 4.

Image Exposure Statistics

To implement the adaptive exposure control of the near infrared light source, exposure information statistics first needs to be performed on data of the collected image. Data that is obtained after statistics is collected is then used for adjusting the near infrared light source. During imaging, a user gives different degrees of attention to exposure conditions of different regions of imaging. To provide the user with more desirable use experience, in the statistics of image exposure information, different weight values should be set according to degrees of attention given by a user to different regions, and weighted averaging is then performed on exposure values of the regions. Generally, the user tends to focus on a central position of an imaging region. In this case, weight distribution is set according to degrees of attention given by the user to imaging regions. The weight of a central region of imaging is increased, and the weights of surrounding regions are relatively reduced. Imaging region division and weight distribution are shown in FIG. 5.

As can be seen from FIG. 5, in the disclosure, the imaging region is divided into 16 regions with equal sizes, and exposure average values Iᵢ of the regions are then calculated, respectively. i is a sequence number of each region. It is set that a weight vector in the foregoing figure is W. In this case, a brightness weighted average value u of the imaging region is calculated by:

$$u = \frac{{\sum }_{i = 1}^{16} {I}_{i} \times {W}_{i}}{{\sum }_{i = 1}^{16} {W}_{i}} . \qquad ( 5 )$$

Automatic Exposure Adjustment and Control

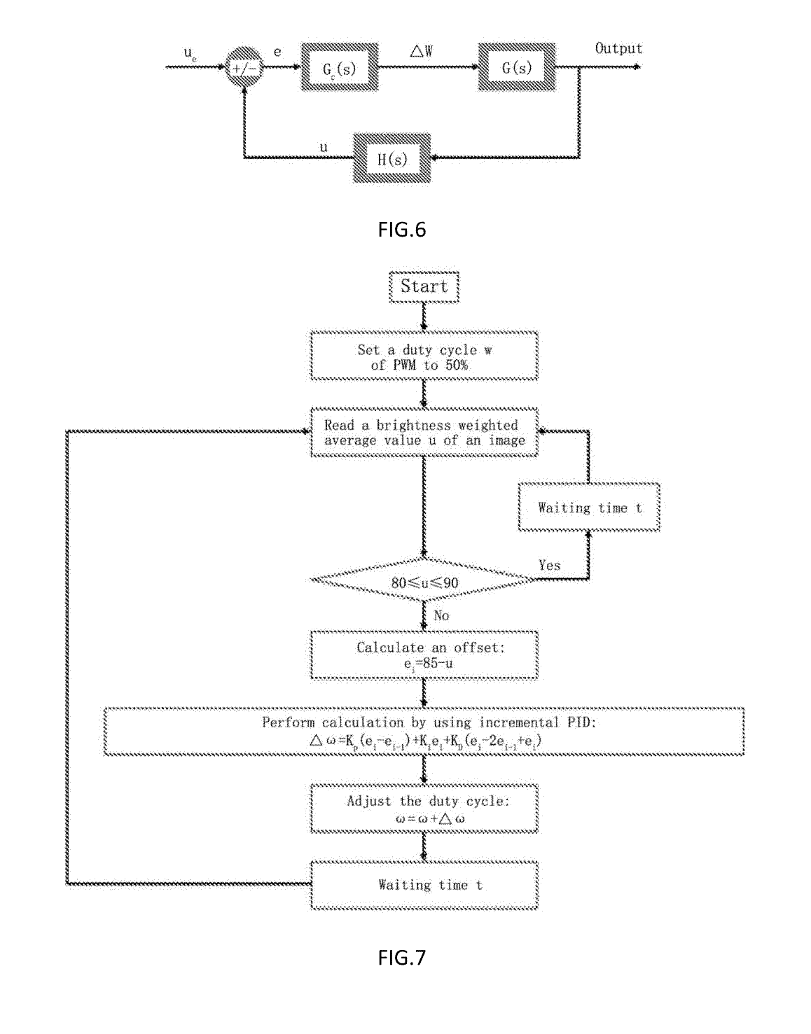

An image exposure assessment module and an automatic exposure adjustment and control module can be considered as one typical closed-loop automatic control system, and can be abstracted as shown in FIG. 6.

In the foregoing figure, uₑ is an ideal average value of image brightness, and G_{c}(s) is a transfer function of a controller. Δw is a control increment, and is an adjustment amount of the illumination brightness of the near infrared light source in the disclosure and is used to adjust the intensity of the near infrared light source, G(s) is a transfer function of a controlled unit and is the near infrared light source in the system, H(s) is a feedback transfer function, and u is the brightness weighted average value of the imaging region introduced above. In the disclosure, the controller is implemented by using a proportional-integral-derivative (PID) automatic control algorithm.

A PID controller is the most widely applied and most stable control algorithm in an actual industrial control process at present. An input/output relationship of the PID controller is shown in the following formula:

$$u ( t ) = {K}_{p} e ( t ) + {K}_{i} {\int }_{0}^{t} e ( t ) dt + {K}_{D} \frac{de ( t )}{dt} , \qquad ( 6 )$$

where Kₚ, Kᵢ, and K_{D} are respectively a proportional coefficient, an integral coefficient, and a differential coefficient. An ideal control effect can be obtained by adjusting the three coefficients. Specifically, the parameter Kₚ is used to control an adjustment speed, the parameter Kᵢ is used to eliminate a steady state error, and the parameter K_{D} is used to improve the dynamic performance^{[34]} of the system. e(t) is an error between a system feedback amount u and a system ideal value uₑ.

According to the characteristics of the control processing system, in the disclosure, real-time image exposure information statistics is used on the programmable logic subsystem, and automatic exposure adjustment and control is performed on the processor subsystem according to statistical data and by using a PID automatic control algorithm. A specific procedure of the control algorithm of the control processing system is shown in FIG. 7.

Contrast Enhancement of a Subcutaneous Venous Image

Differences in the reflection of near infrared light are used to collect a subcutaneous venous image. The contrast of the image is usually relatively low. If the subcutaneous venous image with low contrast is directly projected and displayed, the effect of developing and imaging can fail. Therefore, a contrast enhancement technology for the subcutaneous venous image is a critical technology of the subcutaneous vein developing system, and is used as third-level processing after image data collection in the disclosure, as shown in FIG. 4.

CLAHE-Based Improvement Algorithm

CLAHE is used to minimize the amplification of noise during the enhancement of detailed contrast of an image. A histogram is cut to restrict an enlargement amplitude, that is, a slope of a cumulative distribution function (CDF) is limited. Therefore, the value of a truncation coefficient α of the algorithm needs to be determined by making a compromise between a contrast enhancement effect and a noise suppression degree. To achieve a maximum contrast enhancement effect, the truncation coefficient α needs to have a relatively large value. In this case, the noise suppression degree is reduced. In the disclosure, the original CLAHE is improved according to the characteristics of a venous image. The objective is to further improve a contrast enhancement effect on a venous image and at the same time suppress the amplification of background noise in the image.

To achieve a maximum contrast enhancement effect, improvements are made based on the original CLAHE in the disclosure, and mainly include two aspects: the step of cutting and redistribution for removing a histogram; and the improvement of a CDF mapping function. Specific steps of the CLAHE-based improvement algorithm provided in the disclosure are as follows:

(1) Image Segmentation

The step is consistent with the CLAHE introduced in the foregoing section. In the disclosure, an input image is divided into 4×4 nonoverlapping subblocks.

(2) Statistics of Histograms of the Subblocks

The histogram of each subblock is separately denoted as H_{i,j} (k), where i,j=1, 2, 3, 4. After the statistics of the subblock histograms is collected, the histograms in this method are not cut and redistributed. Instead, a next step is performed.

(3) Calculation of a Hybrid Cumulative Distribution Function (HCDF) of Each Subblock

In the foregoing analysis, the background of the subcutaneous venous image is usually located in low-grayscale level parts, and venous vessels are hidden in middle and high grayscale level parts. Therefore, one threshold Th needs to be determined in the step, and a histogram is divided into two parts. A part that is less than Th is an image background part, and a part that is greater than Th is a region of interest. The adaptive exposure control of the near infrared light source designed in the disclosure can ensure that the average brightness value of the image collected by the system is kept in a stable state. Therefore, the threshold Th used to divide an image background and a region of interest does not need to be dynamically adjusted. To increase an enhancement effect on a region of interest by the algorithm, the amplification of background noise is reduced at the same time. In the disclosure, CDF in an original method is improved, and an HCDF is designed as shown by the following formula:

$${C}_{hb} ( {X}_{k} ) = \{ \begin{matrix}{\sum }_{j = 0}^{k} p ( {X}_{j} ) \times 0.2 , 0 \leq k < Th \\ {C}_{hb} ( {X}_{τ h - 1} ) + {\sum }_{j = Th}^{k} p ( {x}_{j} ) \times {log}_{2} ( j ) , Th \leq k < L\end{matrix} . \qquad ( 23 )$$

The HCDF can achieve different enhancement effects for the image background and a region of interest, where

$$P ( {x}_{k} ) = \frac{{n}_{k}}{W \times l} ,$$

that is, a probability density distribution function (PDF) corresponding to each subblock. It is known that when a slope of a CDF is larger, an enhancement effect on the CDF is stronger. By contrast, when a slope is smaller, an enhancement effect is weaker. The slope of the CDF in the CLAHE algorithm is calculated by:

$$\overline{c} ( {X}_{K} ) = \frac{c ( {X}_{K} ) - c ( {X}_{K - 1} )}{{X}_{K} - {X}_{K - 1}} = \frac{{\sum }_{j = 0}^{k} p ( {x}_{j} ) - {\sum }_{j = 0}^{k - 1} p ( {x}_{j} )}{1} = p ( {x}_{k} ) . \qquad ( 24 )$$

Correspondingly, the slope of the HCDF provided in the disclosure is calculated by:

$$\overline{{c}_{hb}} ( {X}_{k} ) = \frac{{C}_{hb} ( {X}_{k} ) - {C}_{hb} ( {X}_{k - 1} )}{{X}_{k} - {X}_{k - 1}} = \{ \begin{matrix}p ( {x}_{k} ) \times 0.2 , 0 \leq k < Th \\ p ( {x}_{k} ) \times {log}_{2} k , Th \leq k < L\end{matrix} . \qquad ( 25 )$$

When the background part of the image is processed, that is, if the grayscale level is in the range of 0<k<Th,

c̅_̅{̅h̅b̅}̅(Xₖ)=p(xₖ)×0.2<c̅(Xₖ), 0<k<Th (26).

When an image region whose grayscale level is Th≤k<L is processed,

c̅_̅{̅h̅b̅}̅(Xₖ)=p(xₖ)×log₂ k>c̅(Xₖ), Th≤k<L (27).

As can be seen from the above, the enhancement effect of the HCDF on the image background part is weaker than the enhancement effect of the original CDF. That is, the HCDF has a particular suppression effect on the amplification of background noise. In another aspect, the enhancement effect of the HCDF on a region of interest in the image is stronger than the enhancement effect of the original CDF. The enhancement effect of the algorithm is further increased. Before a next step is performed, normalization operation processing further needs to be performed on the HCDF. The HCDF obtained after normalization processing is represented as:

$$T ( {X}_{k} ) = \frac{{C}_{hb} ( {X}_{k} )}{{C}_{hb} ( {X}_{L - 1} )} , 0 \leq k < L . \qquad ( 28 )$$

(4) Establish an Output Mapping Function of Each Subblock

This step is similar to the original CLAHE algorithm. It is set that the HCDF of each subblock obtained in the previous step is T_{i,j}(Xₖ), and i and j are respectively a horizontal sequence number and a vertical sequence number of an image subblock. In this case, an HCDF-based output mapping function is:

Zᵢⱼ(x)=X₀+(X_{L-1}−X₀)T_{i,j}(x)i,j=1,2,3,4 (29).

(5) Pixel Reconstruction Mapping

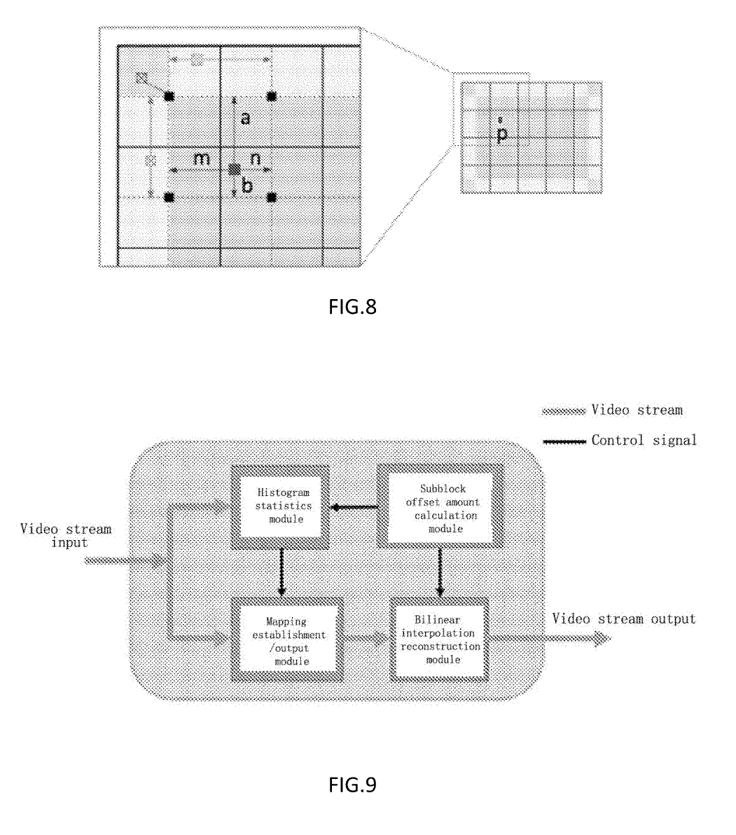

This step is consistent with the original CLAHE algorithm. Based on the output mapping function of each subblock obtained in the previous step, a central position of each subblock is used as a base point, and a bilinear interpolation method is used to reconstruct grayscale values of pixel points in the image, as shown in FIG. 8.

It is set that the pixel point p is located on the upper left side of the subblock (i,j). In this case, a weight value is determined according to a position relationship between the point p and a reference point nearest to the point p, and an eventual weighted result is finally calculated according to the following formula:

$${P}_{out} = \frac{α}{α + b} [ \frac{m}{m + n} {C}_{i , j} ( {P}_{i n} ) + \frac{n}{m + n} {C}_{i - 1 , j} ( {P}_{i n} ) ] + \frac{b}{α + b} [ \frac{m}{m + n} {C}_{i , j - 1} ( {P}_{i n} ) + \frac{n}{m + n} {C}_{i - 1 , j - 1} ( {P}_{i n} ) ] . \qquad ( 22 )$$

Implementation of the Improvement Algorithm

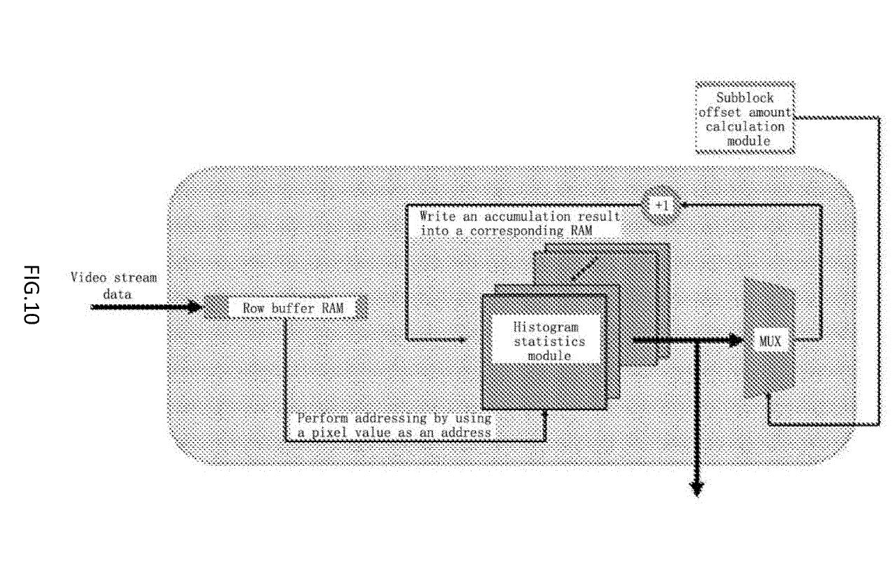

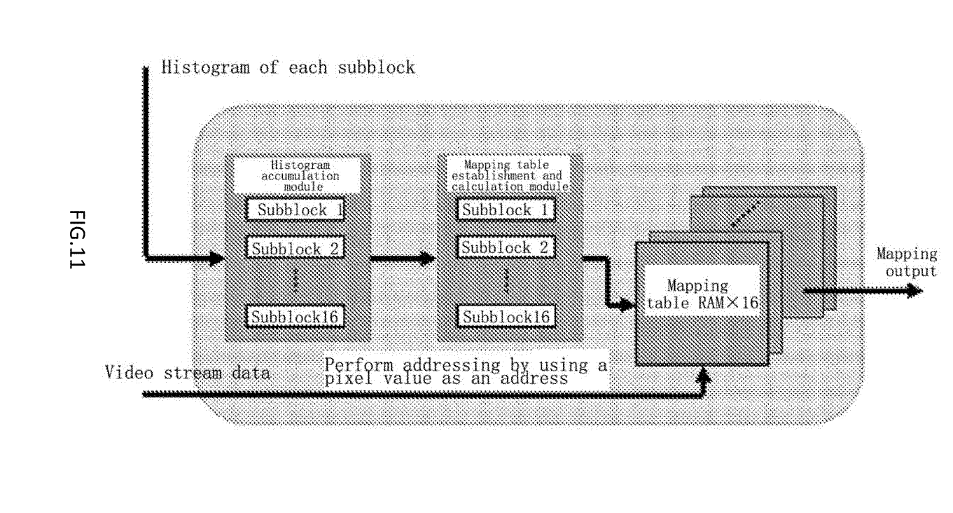

According to the characteristics of the control processing system, an image contrast enhancement module designed in the disclosure can include four submodules according to functional division: a histogram statistics module, a mapping establishment/output module, a bilinear interpolation reconstruction module, and a subblock offset amount calculation module. An overall framework is shown in FIG. 9.

Herein, a design method in which histogram statistics and mapping output are performed synchronously is used. Because a video stream has continuity, histograms of images of adjacent frames having very high similarity. The module is internally designed in a flow-line manner. During an effective field of an n^{th} frame, video stream data enters the histogram statistics module and the mapping establishment/output module at the same time. In this way, histogram statistics and a mapping table lookup output operation are performed at the same time. Data that flows from the mapping establishment/output module passes through the bilinear interpolation module for pixel reconstruction. During the blanking of the field of the n^{th} frame, the transmission of the video stream data stops. In this case, the mapping establishment/output module reads completed histograms from the histogram statistics module to establish a mapping table for use by mapping output of a next frame of image. Video stream data of an (n+1)^{th} frame uses the mapping table established during the blanking of the field of the n^{th} frame for mapping output.

(1) Subblock Offset Amount Calculation Module

The module performs positioning and tracking on a current pixel of a video stream input to calculate sequence numbers i and j of a subblock in which the current pixel is located and relative coordinates m, n, a, and b of the pixel in the subblock. The histogram statistics module and the bilinear interpolation module complete operations such as data selection and weight value calculation according to the statistical information.

(2) Histogram Statistics Module

The module has one row buffer RAM and 16 histogram statistics RAMs. A specific architecture is shown in FIG. 10. During an effective field, the video stream data is first written into the row buffer RAM. One pixel value is written in each clock period. After one row of data is filled, transmission of a first-level video stream is paused. In this case, the row buffer RAM starts to be read to perform histogram statistics. In combination with a sequence number of a subblock given by the subblock offset amount calculation module, calculation results are input into a histogram statistics RAM corresponding to the subblock. After a pixel value is read from the row buffer RAM, data is read from a histogram statistics RAM of a corresponding subblock in a next clock period and is accumulated. Then, in a next clock period, an accumulation result is rewritten into the histogram statistics RAM of the corresponding subblock. A total of three clock periods is needed for the three steps. After data in the row buffer RAM is read, a previous-level module restarts transmission of a data in a next row.

After one frame of image has been transmitted, a field blanking time is entered. In this case, statistics of each subblock histogram is completed. The mapping establishment/output module reads data from the histogram statistics RAM to establish a mapping table corresponding to each subblock. After the mapping table has been established, the histogram statistics RAM is reset and then waits for data of a next effective field.

(3) Mapping Establishment/Output Module

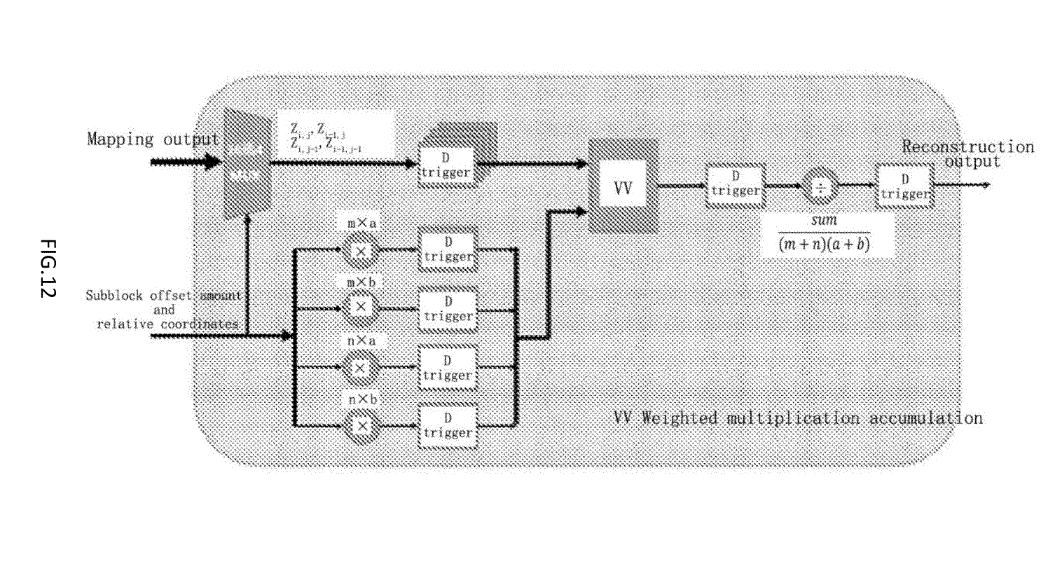

The architecture of the mapping establishment/output module is shown in FIG. 11. The module has a histogram accumulation module, a mapping table establishment and calculation module, and 16 mapping table RAMs that correspond to 16 subblocks respectively. During an effective field of a video, the mapping establishment/output module receives the video stream data and perform lookup and output of a mapping table RAM. During field blanking of the video, the histogram accumulation module reads data from the histogram statistics RAM to perform accumulation, and a result is transferred to a mapping table calculation module. The mapping table calculation module establishes a mapping table based on the HCDF provided herein. Specifically, multiplication operations of p(Xⱼ)×0.2 and p(xⱼ)×log₂(j) in Formula (23) are implemented in a look up table (LUT) manner. In this way, the processing speed is increased and multiplier resources are saved. The data processing of the histogram accumulation module and the mapping table calculation module is designed in a flow-line manner. Eventual calculation results are written into corresponding mapping table RAMs. The calculation of subblocks in the modules are performed in parallel and do not affect each other. In this way, the processing speed of the algorithm can be greatly increased and the real-time performance requirement of the system is satisfied.

(4) Bilinear Interpolation Module

During an effective field, the module reads data from a mapping table RAM and performs bilinear interpolation reconstruction output. Before bilinear interpolation is implemented, Formula (22) is simplified first to facilitate hardware implementation. The simplified formula is shown as follows:

$${P}_{out} = \frac{{maz}_{i , j} ( {P}_{i n} ) + {naz}_{i - 1 , j} ( {P}_{i n} ) + {mbz}_{i , j - 1} ( {P}_{i n} ) + {nbz}_{i - 1 , j - 1} ( {P}_{i n} )}{( m + n ) ( α + b )} . \qquad ( 30 )$$

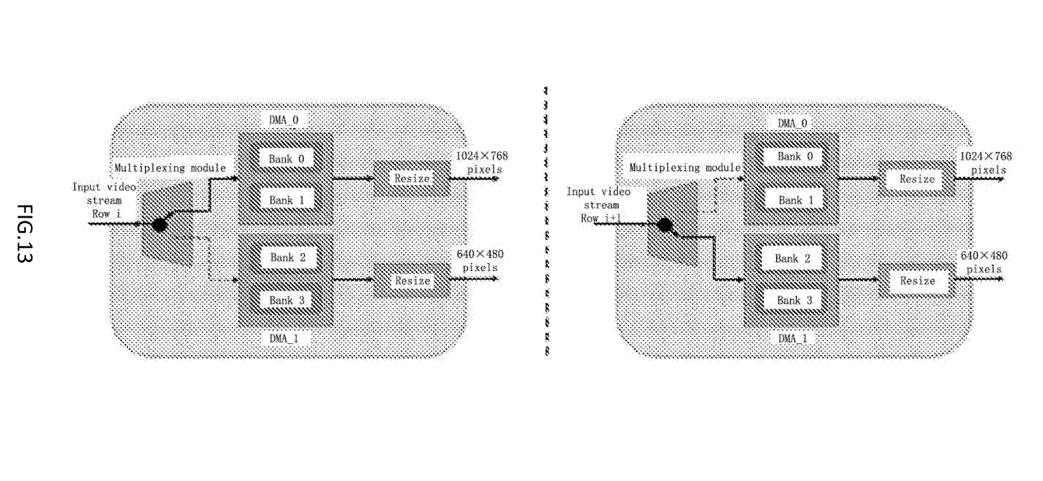

The module is related to a plurality of operations such as data selection, weight value calculation, interpolation rule selection, and weighted multiplication accumulation. Therefore, a multi-level flow-line design method is used herein to enable the module to achieve a maximum data throughput and optimal calculation performance. A flow-line framework of bilinear interpolation is shown in FIG. 12.

In the figure, the bilinear interpolation module uses a four-level flow-line design. One flow-line is further embedded in a weighted multiplication accumulation module.

Two-Way Synchronous Display (Video Source Multiplexing)

As shown in FIG. 4, a video source multiplexing module is used as final-level processing before video output in the disclosure and is used to implement a function of two-way synchronous display output of the projection imaging element and the display screen.

The function of two-way synchronous display is one innovative design of the disclosure. The function enables the system to project an image by using the projection imaging element and perform synchronous display on the display screen. This can assist medical staff in making a double check on the position of a vein to improve puncture precision and adapt to different operation habits of the medical staff.

In FIG. 13, the modular parts can all be interconnected via an AXI bus interface to implement a specific procedure as follows:

(1) During an effective field, video stream data continuously flows into a multiplexing module.

(2) The multiplexing module can perform an interlaced switching mode. Data of an input video stream is respectively switched to different paths. For example, an i^{th} row of data of the input video stream flows into a direct memory access (DMA) 0 module, and a next row of data is switched to a DMA1 module.

(3) The DMA0 module and the DMA1 module receive video stream data of a previous level. The video stream data is written into two different banks in the memory by using DMA to be read by the projection imaging element and the display screen respectively. A pingpong operation design is further used in each module. In this way, video buffering and video reading can be performed at the same time, and next-level processing and previous-level processing are separated.

(4) Video buffer data in corresponding banks is read respectively in two paths by using DMA, and is then scaled by using an image scaling module to an appropriate output resolution size.

(5) Output video stream data in two paths flows respectively to the projection imaging element driving circuit module and the display screen driving circuit module to implement two-way synchronous display of a single video source.

1. A control processing system, comprising
a processor subsystem, and
a programmable logic subsystem interconnected to the processor subsystem via a high-bandwidth bus,
wherein the high-bandwidth bus is an Advanced eXtensible Interface (AXI) bus including AXI-Lite or AXI-Stream,
and wherein the control processing system is in signal communication with a visible light source driving circuit, a near infrared light source driving circuit, a projection imaging element driving circuit, a near infrared imaging element driving circuit, a display screen driving circuit, and a user control interface.

2. The control processing system according to claim 1, wherein the visible light source driving circuit, the near infrared light source driving circuit, the projection imaging element driving circuit, the near infrared imaging element driving circuit, the display screen driving circuit, and the user control interface are in signal communication with the control processing system, to implement data collection and projection imaging of a near infrared image of a subcutaneous venous vessel; and the control processing system is responsible for image data processing and system control.

3. The control processing system according to claim 1, further comprising an image data collection module, an image cutting and scaling module, an image exposure statistics module, an image contrast enhancement module, a video source multiplexing module, a projection output module, a display screen output module, an automatic exposure adjuster and controller module, an image exposure assessment module, a system parameter control module, and a memory module, wherein the modules are in signal communication with each other.

4. An image processing method using a control processing system, comprising:
collecting data on a near infrared image of a subcutaneous venous vessel, and performing cutting, scaling, and offset adjustment processing on a collected image;
adjusting a near infrared light source in a subcutaneous vein developing device system according to an exposure condition of the collected image, to provide a stable and appropriate image exposure status for subsequent image enhancement processing, wherein the adjusting a near infrared light source in a subcutaneous vein developing device system comprises image exposure statistics, image exposure assessment, and automatic exposure adjustment and control of the light source;
enhancing the contrast of the image; and
performing further processing on a resultant image to be output, to implement two-way synchronous display output of a projection imaging element and a display screen.

5. The image processing method according to claim 4, wherein during collecting data on a near infrared image of a subcutaneous venous vessel,
sizes of actual coverage regions of a collection lens and a projection lens are measured respectively according to a projected image, and a position of the coverage region of the projection lens relative to the coverage region of the collection lens is measured, to perform cutting, scaling, and offset adjustment processing on the collected image.

6. The image processing method according to claim 4, wherein in the image exposure statistics different weight values are set according to degrees of attention given by a user to different regions and then performing weighted averaging on exposure information of each region, and next, comparison and assessment are performed on obtained image exposure information and automatic exposure adjustment and control of the near infrared light source is implemented.

7. The image processing method according to claim 4, wherein the enhancing comprises a transform domain-based method, a histogram equalization method, or various improvement methods derived therefrom.

8. The image processing method according to claim 7, wherein the improvement methods comprise global histogram equalization, or brightness preserving bi-histogram equalization, or Sigmoid function-based bi-histogram equalization, or contrast limited adaptive histogram equalization (CLAHE).

9. The image processing method according to claim 4, further comprising processing an output video by using a time-division multiplexing method, to implement two-way synchronous display output of the projection imaging element and the display screen.
